(19) United States
(12) Patent Application Publication
(10) Pub. No.: US 2012/0130477 A1
(43) Pub. Date: May 24, 2012

(54) VASCULAR PROSTHESIS AND RELEVANT METHOD FOR REALISING THEREOF

(76) Inventor: Massimiliano Gessaroli, Cattolica (RN) (IT)

(21) Appl. No.: 13/261,018

(22) PCT Filed: Apr. 7, 2010
(86) PCT No.: PCT/IT2010/000146

§ 371 Date: Dec. 28, 2011

(30) Foreign Application Priority Data
May 21, 2009 (IT) RN2009A000023
Nov. 25, 2009 (IT) RM2009A000620

(51) Int. Cl. A61F 2/82

(52) U.S. Cl. 623/1.15

(57) ABSTRACT

The present invention relates to a tubular shaped vascular prosthesis (10; 11), mainly extending along the longitudinal dimension which has a main portion (20; 21), having a first (201; 201) and a second end (202; 212), and at least an end portion (30; 31) having an elasticity along its transverse direction with respect to its longitudinal dimension, said end portion (30; 31) being coupled with one of the first (201; 211) or second end (202; 212) of the main portion (20; 21) and can be coupled to the wall of a blood vessel.

The present invention further relates to a method for realising a vascular prosthesis (10; 11).

The present invention relates to an improved vascular prosthesis and relevant method for realising thereof.

More specifically, the invention concerns a vascular prosthesis that can better conform to the pulsation of blood vessels, thus reducing risk that the same can detach from the blood vessel to which it is fixed, and mainly that do not generate, where it is fixed to the blood vessel, a lumen narrowing, as it always occurs with present prosthesis, thus losing its functionality.

As it is well known, prosthesis for realising bypass or for haemodialysis have been employed in vascular surgery.

Present synthetic vascular prosthesis are mainly employed for replacing obstructed or expanded arterial peripheral vessels (in case of aneurysms) or to realise the so called prosthetic vascular access for haemodialysis. Said prostheses are mainly comprised of ePTFE (expanded polyfluorotetraethylene). Said vascular prosthesis are realised with different shapes. Particularly, they can be rectilinear or conical, with different thicknesses (standard or thin), with or without an enlarged end portion (also known as cap), with the wall impregnated or otherwise bond with heparin.

However, although such an evolution occurred during the last years, present synthetic prosthesis are in any case characterised by a high probability of clogging, mostly due to progressive distal anastomosis (term indicating the point where two vessels are communicated or a prosthesis is connected with a vessel) narrowing (phenomenon also known as stenosis) in peripheral bypass or of venous anastomosis in prosthetic vascular accesses.

Said vessel narrowing is caused by an enormous proliferation of smooth muscular fibre cells, due to increase of activity of fibroblasts present in the vessel wall of the juxta-anostomotic portion (i.e. the portion close to the anastomosis), which are stressed by wall shear stress, i.e. stress to which vessel wall is subjected due to the different elastic response of the same with respect to the prosthetic wall (different compliance) to the haematic pulsating wave.

Many studies, both histopathologic and biomechanical studies, have demonstrated that main reasons of said anostomotic hyperplasia comprise surgical trauma, biocompatibility of synthetic prosthesis, as well as different local mechanical stresses on vessel wall, such as endo-lumen hypertension, flow vortex of some areas and vibrations of the vessel walls caused by flow within connection between vessels (juxta-anostomotic connection).

A first attempt for reducing said phenomenons has been introduced by W.L. Gore & Associates Inc., which produced a prosthesis made up of longitudinally stretchable ePTFE caused by cardiac sisto-diastolic mechanical stress, thus trying reproducing kinetic of an arterial or venous vessel. Said solution, even inducing a small improvement of life of said prosthesis, has not solved the above mentioned problems. It is further possible explaining that this result by the fact that, during the modification of the arterial pressure wave during the cardiac sisto-diastolic phases, vessels generically respond with transverse rather than longitudinal pulsation and expansion, and thus also this kind of prosthesis has a remarkable different compliance with respect to original vessels.

A further attempt according to the prior art is described in U.S. Pat. No. 6,589,278, wherein it is suggested adding a cup to the synthetic prosthesis, said cup being comprised of autologous venous material of the same patient, interposed between the prosthetic segment and the vessel to be anastomised. However, in this case too results obtained, although better than those obtained without venous cup, do not fully solve the above mentioned problems, since shear stress reproduces between prosthesis and the same venous cup.

Other authors attempted reducing incidence of anostomotic hyperplasia, directly creating an ePTFE prosthesis, enlarged at one end (cup) in order to improve flow hemodynamic at the anostomotic level, but also in this case the expected result was not conform to expectations since, in any case, remains an elasticity biomechanical difference of vessel with respect to prosthesis, which, under the systolic wave, determines a continuous micro trauma at the sutra level on the same vessel.

Therefore, object of the present invention is that of suggesting an improved vascular prosthesis able limiting phenomenon of anostomotic myointimal hyperplasia of vessels in contact point between prosthesis and vessel, thus obtaining a longer life of the same prosthesis.

These and other results are obtained according to the invention providing a prosthesis that can absorb stresses due to arterial pressure waves during sisto-diastolic cardiac phases in suture point of the same prosthesis on vessel wall also along a transverse direction.

It is therefore specific object of the present invention a tubular shaped vascular prosthesis, mainly extending along the longitudinal dimension, characterised in that it comprises a main portion, having a first and a second end, and at least an end portion having an elasticity along its transverse direction with respect to its longitudinal dimension, said end portion being coupled with one of said first or second end of said main portion and can be coupled to the wall of a blood vessel.

Always according to the invention, said prosthesis can comprise two end portions, respectively coupled with said first and second end.

Still according to the invention, said at least one end portion and said main portion can be realised as a single piece.

Furthermore, according to the invention, said at least one end portion can be coupled to the relevant end of said main portion for suture and/or gluing and/or welding, particularly heat welding.

Advantageously, according to the invention, said main portion can have elasticity along said longitudinal direction.

Always according to the invention, said main portion can have a transverse elasticity with respect to said longitudinal direction.

Still according to the invention, said prosthesis can be of the type suitable for peripheral bypass and/or haemodialysis.

Furthermore, according to the invention, said prosthesis can be comprised of expanded polytetrafluoroetyhlene, of the stretch, standard and/or intering and/or Propaten type and/or of any other elastic prosthetic material.

Advantageously, according to the invention, said at least one end portion can be chosen on the basis of the following groups: rectilinear portion; conical portion; rectilinear bevelled portion; conical bevelled portion.

It is further object of the present invention a method for realising a vascular prosthesis, comprising the following steps:

(a) providing a main vascular prosthesis;

(b) providing a portion of a further vascular prosthesis having elasticity along said longitudinal dimension all along its extension;

(c) longitudinally sectioning said further portion of vascular prosthesis of said step (b);

(d) transversely folding and coupling said portion of sectioned vascular prosthesis of said step (c); and

(e) coupling said portion of said further vascular prosthesis with said main vascular prosthesis.

Always according to the invention, in said step (d), said portion of a further vascular prosthesis is fold and transversely sutured, so as to obtain said end portion chosen on the basis of one of the following groups: rectilinear portion; conical portion; rectilinear bevelled portion; conical bevelled portion.

Still according to the invention, coupling in said steps (d) and/or /e) occurs by suture and/or gluing and/or welding, particularly heat welding.

Advantageously, according to the invention, said main vascular prosthesis can have elasticity along its longitudinal direction and said portion of said vascular prosthesis portion is obtained by a section of a segment of said main vascular prosthesis.

Present invention will be now described for illustrative but not limitative purposes according to its preferred embodiments, with particular reference to the figures of the enclosed drawings, wherein:

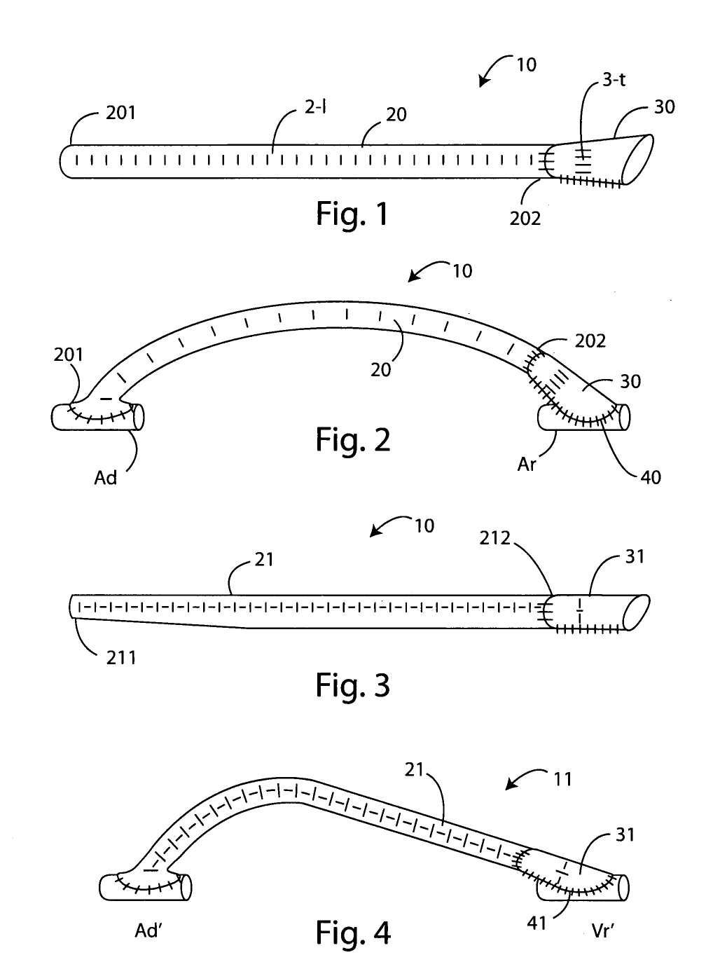

FIG. 1 shows a vascular prosthesis for peripheral bypass according to the present invention;

FIG. 2 shows vascular prosthesis of FIG. 1 connecting two arterial vessels;

FIG. 3 shows a vascular prosthesis for haemodialysis according to the present invention;

FIG. 4 shows vascular prosthesis of FIG. 1 connecting an arterial vessel and a venous vessel;

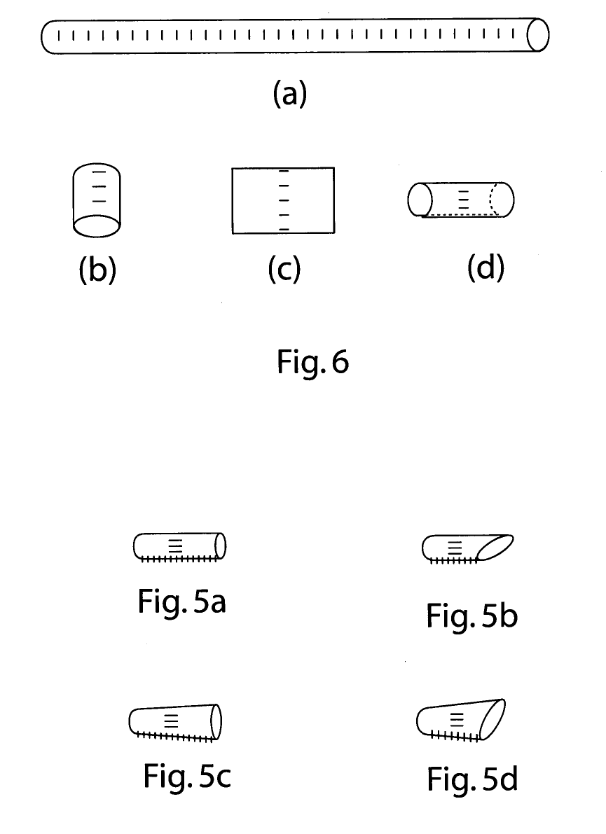

FIGS. 5a-5d show a plurality of embodiments of a part of prosthesis according to the invention; and

FIG. 6 shows steps of realisation of a vascular prosthesis according to the present invention.

Similar parts in various figures will be indicated by the same reference numbers.

Making reference to FIG. 1, it is observed a vascular prosthesis 10 for peripheral bypass according to the invention, comprising a main portion 20, extending longitudinally, a first and a second ends 201 and 202. Said main portion 20 is graphically defined in the figure by longitudinal lines 2-l, indicating longitudinal direction according to which said vascular prosthesis 10 portion can be elastically extended.

Vascular prosthesis 10 also comprises an end portion 30, fixed at the second end 202 of said main portion 20. End portion 30 is graphically identified by transverse lines 3-t, showing that said end portion 30 is elastic according to a direction which is transverse with respect to the direction of the main portion 20, directed along its longitudinal direction. Said end portion 30 can be coupled to the wall of a blood vessel.

FIG. 2 shows connection of two arterial vessels by vascular prosthesis 10 for peripheral bypass described in the above. Particularly, vascular prosthesis 10 connects a donor artery Ad and a receiving artery Ar. Longitudinal elasticity permits to main portion 20 of prosthesis longitudinally elongating under cardiac sisto-diastolic mechanic stresses generated by donor artery Ad. Instead, end portion 30 permits limiting possible micro-traumas along suture line 40 connecting between end portion 30 of vascular prosthesis 1 and receiving vein Vr wall. Said micro traumas are always due to cardiac sisto-diastolic mechanical stresses, creating a standard transverse (or radial) pulsating movement of said receiving artery Ar thanks to its transverse elasticity, end portion 30 being able to conform its cross-section, thus following said alternate movement.

FIG. 3 shows a vascular prosthesis 11 for haemodialysis always comprising a main portion 21, wherein first end 211 has a narrowing or tapering, just for haemodialysis, while at the second end 212 it is coupled an end portion 31, having an elasticity which is transverse with respect to elasticity of main portion 21. In the present embodiment, said end portion 31 is coupled with said second end 121 of said main portion 21 by suture.

FIG. 4 shows connection of a donor artery Ad′ and a receiving vein Vr′ by vascular prosthesis 11 for haemodialysis as described in the above. As it can be observed, in this case too, said end portion 31 is surgically coupled with an opening obtained on the wall of said receiving vein Vr′ along the suture line 41. Operation of vascular prosthesis 11 it is similar to the one described for vascular prosthesis 10.

FIGS. 5a-5d show different embodiments of end portion 30 or 31 of vascular prosthesis 10 or 11. More specifically:

FIG. 5a shows a rectilinear-type end portion 30 or 31;

FIG. 5b shows a rectilinear bevelled-type end portion 30 or 31;

FIG. 5c shows a conical-type end portion 30 or 31;

FIG. 5d shows a conical bevelled-type end portion 30 or 31.

Finally, FIG. 6 shows steps of a method for realising a vascular prosthesis 1 according to the invention, namely:

(a) providing a main vascular prosthesis, for example for peripheral bypass or for haemodialysis, having a longitudinal elasticity all along its extension;

(b) providing a portion of a further vascular prosthesis having elasticity along said longitudinal dimension all along its extension;

(c) longitudinally sectioning said further portion of vascular prosthesis;

(d) transversely folding and suturing said portion of said further vascular prosthesis; and

(e) suturing said portion of said further vascular prosthesis with said main vascular prosthesis.

Different modifications of the prosthesis according to the invention can be taken into consideration, such as those wherein said main portion 20 does not have elastic properties, or it has an elasticity transversal with respect to its longitudinal direction.

Above method is an hand-craft assembling of a prosthesis having a longitudinal elasticity, for example using standard stretch PTFE prosthesis manufactured by W.L. Gore, in its standard, Intering (with PTFE intraparietal reinforcing rings), Propaten (PTFE with covalent bond with Heparin), with an end portion (cap) comprised of a segment of the same kind of prosthesis. However, it is possible realising prosthesis also by a material different with respect to PTFE or like, with transverse elasticity, which is longitudinally sectioned and then knit transversely, having at the end a transverse elasticity (synthetic cap for vascular prosthesis) that can be compared with the one of a vessel.

Solution providing a direct knitting of tissue in order to generate cap and its coupling to prosthesis when it is the method is available, but as a further modification, it is possible employing different methods such as gluing or welding, particularly heat welding. Said end portion or cap can be realised in such a way to make a rectilinear prolongation of prosthesis or it can be conical.

Furthermore, it must be taken into consideration that vascular prosthesis 10 or 11 thus assembled can be used in vascular fittings for haemodialysis, anatomising part of cap or end portion by efferent nein or it can be used in peripheral bypass anatomising part of the cap with receiving distal artery.

Present invention can also be integrally realised to one or both transverse elasticity ends.

In a further embodiment, vascular prosthesis 10 and 11 according to the invention can be manufactured and realised as a single piece.

The present invention has been described for illustrative, but not limitative purposes, according to its preferred embodiments, but it is to be understood that variations and/or modifications can be introduced by those skilled in the art without departing from the relevant scope, as defined in the enclosed claims.

1. Tubular shaped vascular prosthesis (10; 11), mainly extending along the longitudinal dimension, said vascular prosthesis (10; 11) comprising
a main portion (20; 21), having a first (201; 201) and a second end (202; 212), and
at least one end portion (30; 31)
characterised in that
said at least one end portion (30; 31) has an elasticity along its transverse direction with respect to its longitudinal dimension, is coupled with one of said first (201; 211) or second end (202; 212) of said main portion (20; 21), and can be coupled to the wall of a peripheral venous or arterial blood vessel, and has a cylindrical or troncoconical shape,
so that said at least one end portion (30; 31) is capable to conform its cross-section to the alternate movement that can be created by a transverse or radial pulsating movement of said vessel to which it can be coupleable.

2. Vascular prosthesis (10; 11) according to claim 1, characterised in that it comprises two end portions (30; 31), respectively coupled with said first (201; 211) and second end (202; 212).

3. Vascular prosthesis (10; 11) according claim 1, characterised in that said at least one end portion (30; 31) and said main portion (20; 21) are realised as a single piece.

4. Vascular prosthesis (10; 11) according claim 1, characterised in that said at least one end portion (30; 31) is coupled to the relevant end of said main portion (20; 21) for suture and/or gluing and/or welding, particularly heat welding.

5. Vascular prosthesis (10; 11) according to claim 1 characterised in that said main portion (20; 21) has elasticity along said longitudinal direction.

6. Vascular prosthesis (10; 11) according to claim 1 haracterised in that said main portion (20; 21) has a transverse elasticity with respect to said longitudinal direction.

7. Vascular prosthesis (10; 11) according to claim 1 characterised in that said it is of the type suitable for peripheral bypass and/or haemodialysis.

8. Vascular prosthesis (10; 11) according to claim 1 characterised in that said it is comprised of expanded polytetrafluoroetyhlene (PTFE), of the stretch, standard and/or intering and/or Propaten type and/or of any other elastic prosthetic material.

9. Vascular prosthesis (10; 11) according to claim 1 characterised in that said at least one end portion (30; 31) is chosen on the basis of the following groups:
rectilinear portion;
conical portion;
rectilinear bevelled portion;
conical bevelled portion.

10. Method for realising a vascular prosthesis (10; 11) as defined in claim 1, comprising the following steps:
(a) providing a main vascular prosthesis;
(b) providing a portion of a further vascular prosthesis having elasticity along said longitudinal dimension all along its extension;
(c) longitudinally sectioning said further portion of vascular prosthesis of said step (b);
(d) transversely folding and coupling said portion of sectioned vascular prosthesis of said step (c); and
(e) coupling said portion of said further vascular prosthesis with said main vascular prosthesis.

11. Method for realising a vascular prosthesis (10; 11) according to claim 10, characterised in that in said step (d), said portion of a further vascular prosthesis is fold and transversely sutured, so as to obtain said end portion chosen on the basis of one of the following groups:
rectilinear portion;
conical portion;
rectilinear bevelled portion;
conical bevelled portion.

12. Method for realising a vascular prosthesis (10; 11) according to claim 10, characterised in that coupling in said steps (d) and/or /e) occurs by suture and/or gluing and/or welding, particularly heat welding.

13. Method for realising a vascular prosthesis (10; 11) according to claim 10, characterised in that said main vascular prosthesis has elasticity along its longitudinal direction and said portion of said vascular prosthesis portion is obtained by a section of a segment of said main vascular prosthesis.
